(19) United States
(12) Patent Application Publication
(10) Pub. No.: US 2016/0157512 A1
(43) Pub. Date: Jun. 9, 2016

(54) Wildlife Nutritional Supplementation Methods and Compositions

(71) Applicant: David R. Fuhr, New Boston, MO (US)

(72) Inventor: David R. Fuhr, New Boston, MO (US)

(21) Appl. No.: 15/040,172

(22) Filed: Feb. 10, 2016

Related U.S. Application Data
(63) Continuation of application No. 13/418,773, filed on Mar. 13, 2012.
Application No. 13/418,773 is a continuation-in-part of application No. 13/022,872, filed on Feb. 8, 2011, now abandoned.
(60) Provisional application No. 61/453,266, filed on Mar. 16, 2011.
(60) Provisional application No. 61/303,127, filed on Feb. 10, 2010.

(51) Int. Cl. A01N 59/20; A61K 31/355; A61K 31/4188; A61K 31/519; A61K 31/455; A61K 31/197; A61K 31/51; A61K 31/714; A61K 31/525; A61K 31/4415; A61K 31/375; A61K 33/18; A61K 33/10; A61K 33/34; A61K 33/26; A61K 33/08; A61K 33/30; A61K 9/00; A61K 31/20; A61K 31/593

(52) U.S. Cl. CPC A23K 1/1603; A61K 31/20; A61K 31/593; A61K 31/355; A61K 31/4188; A61K 31/519; A61K 31/455; A61K 31/197; A61K 31/51; A61K 31/714; A61K 31/525; A61K 31/4415; A61K 31/375; A61K 33/18; A61K 33/10; A61K 33/34; A61K 33/26; A61K 33/08; A61K 33/30; A61K 9/0056; A23K 1/1753; A23K 1/1755; A23K 1/1758; A23K 1/17

(57) ABSTRACT

This invention relates to wildlife nutritional supplementation compositions and methods of use thereof for providing enhanced feeding efficiency and restoring a ruminant's self-generation of water soluble vitamins.

### CROSS-REFERENCE TO RELATED APPLICATIONS

This application is a continuation of U.S. application Ser. No. 13/418,773, filed Mar. 13, 2012, which claims the benefit of U.S. Provisional Application No. 61/453,266 filed Mar. 16, 2011 and is a continuation-in-part of U.S. application Ser. No. 13/022,872, filed Feb. 8, 2011, which claims the benefit to U.S. Provisional Application No. 61/303,127, filed Feb. 10, 2010, which references are herein incorporated in its entirety.

### FIELD OF THE INVENTION

The invention pertains to the field of wildlife nutritional supplementation compositions and methods of use thereof. More particularly the invention pertains to a ruminant feed supplement providing food grade vitamins and minerals to promote and/or restore a ruminant's self-generation of water soluble vitamins. In addition, the wildlife nutritional supplementation compositions provide an enhanced immune-system, promote peak growth and control insect infestations. Methods for using the wildlife nutritional supplementation compositions are further disclosed.

### BACKGROUND OF THE INVENTION

Supplementing the nutritional deficiencies in range rudiment animals, such as deer, elk and the like, is known in the art. Wildlife nutritional supplements are frequently placed near wildlife trails or other areas frequented by such wildlife. Wildlife nutritional supplements have gained increasing popularity as a means to enhance the health and life span of rudiment animals, such as for improved hunting and gaming. There remains a need for superior products and methods of making the same as a result of the reduction of wildlife habitat. For example, the expansion of human development leaves a noticeable impact on the health and vitality of wild ruminant animals often as a result of decreased food supply for such animals. Nutritional supplementation provides a means for improving the animals' reproduction, weight, antler size, and susceptibility to disease and parasites. Examples of commercially available brands and formulations are described in U.S. Pat. No. 6,572,903, which is incorporated herein by reference in its entirety.

The macronutrients primarily accredited with impacting wildlife health and sustainability includes calcium, phosphorus, potassium, sulfur, sodium, chloride and magnesium. The beneficial effects of these minerals are well known, such as: calcium aiding in the growth of bones, teeth and antlers and is important in the function of muscles and nerves; phosphorus aiding in the growth of bones, teeth and antlers (along with reacting with calcium to maximize such growth effects), enhancing energy metabolism and enzymation as well as proper protein utilization; potassium is integral in the function of nerves, enzyme processes, as well as mineral and water balance in feed intake and digestion; sulfur is an essential component of some proteins and promotes optimum growth and overall health; sodium is vital to the function of muscles and nerves and also maintains water balance; chloride of sodium forms hydrochloric acid in the abomasums which aids in protein breakdown, wherein salt is also critical for overall health and maintaining a stable water balance; and magnesium is an important component is almost all body processes and promotes antler development along with reacting with calcium.

There are also several trace minerals and vitamins known to be essential for maintaining a healthy wildlife, including for example the trace minerals manganese, copper, zinc, iodine, selenium, cobalt and iron, and the vitamins A, B, D, C and E. The well-known beneficial effects of these trace minerals include the following: manganese is essential for good bone development and feed utilization as it impacts metabolism and works as a synergistic medium for calcium, phosphorus, magnesium and B1 vitamins; copper is needed for blood and feed utilization and is perhaps one of the most important enzymes for the control of free radical proteins; zinc provides an important influence on the rate of nutrient absorption, wound healing and general immune system functions as well as assisting in reactions with vitamin B12, calcium and phosphorus; iodine is a vital component of the thyroid hormone which controls body temperature and rate of metabolism; selenium is needed for growth and reproduction and is also involved in enzymatic systems and interacts with vitamin E in synergistic and cooperative manner within metabolism; cobalt is a necessary component of vitamin B12 and enzymes that digest feed; and iron is a critical component of red blood cells.

The beneficial effects of various vitamins for use with wildlife nutritional supplementation compositions are known to include the following: vitamin A is necessary to support bone and teeth growth, vision, reproduction, is involved in bone development and antler growth, and aids in controlling infections; vitamin D is necessary for the mineralization of bone development and antler growth and maintains proper functioning of muscles, nerves, blood clotting and cellular growth; and vitamin E is a component of the enzyme system, acting as an antioxidant at the cellular level acting to protect against the damaging effects of free radicals as well as functioning in selenium metabolism. Vitamins A, D and E are referred to as fat soluble vitamins and must be processed through the small intestinal track of wildlife.

In addition to fat soluble vitamins, water soluble vitamins, including vitamins B and C are known to benefit wildlife. Vitamin C or ascorbic acid is important in the hydroxylation of collagen which in its absence is inadequately hydroxylated and produces the skin lesions and blood vessel weaknesses (such as those characteristic of the deficiency disease known as scurvy) and is further recognized for its functionality in developing a functional immune system, wherein deficiencies are known to cause weight loss, retarded growth, weak bones, loose teeth and weakened immune system.

The various B vitamins are often referred to as “B series” vitamins, which include the following groups: vitamin B-1 (thiamin), vitamin B-2 (riboflavin), vitamin B-3 (niacin), vitamin B-5 (pantothenic acid), vitamin. B-6 (pyridoxine hydrochloride or pyridoxal), vitamin B-7 (biotin or vitamin H), vitamin B-9 (folic acid), and vitamin B-12 (cobalamin). The B series vitamins are known to have the following beneficial effects for use with wildlife nutritional supplementation: vitamin B-1 acts as a catalyst in carbohydrate metabolism enabling carbohydrates to release energy while also serving as a natural insect repellent; vitamin B-2 (riboflavin) acts as a critical cofactor or coenzyme in the metabolism of fats, carbohydrates and amino acids, the deficiency of which causes skin and corneal lesions; vitamin B-3 is a critical element for cellular respiration and the nervous system and is essential for the metabolism of carbohydrates and fats; vitamin B-5 builds new body cells, promotes the utilization of other vitamins and is required in the metabolism of fat, protein and carbohydrates; vitamin B-6 (pyridoxal) is heavily involved in a majority of bodily functions, aids in building the immune system and is required in the synthesis and metabolism of protein and amino acids while also supporting the formation of red blood cells; vitamin B-12 (cobalamin) is critical to normal nerve cell activity, DNA replication and the development of red blood cells and interacts with folic acid and zinc; biotin (also known as vitamin B-7 or H) is a critical cofactor in the metabolism of carbohydrates, proteins and fats while also aiding in antler growth and acting as a multiplier for other B vitamins; and vitamin B-9 (folic acid or tetrahydrofolate) is important for the synthesis of nucleic acids and overall health of ceroids for normal growth and development, including cell replication and cell division, including nerve cell development in fetal fawns or elk calves.

The importance of the above-mentioned minerals, trace minerals and vitamins have been used in a variety of nutritional products to enhance the health and vitality of wildlife. Additionally, various products are available to increase insect repellency, improve palatability to enhance an animal's consumption of the product, and prevent parasitic and bacteria infections.

Despite the numerous health-related claims made by various wildlife supplement products, there remain numerous drawbacks to currently-available formulations as well as methods of use in failing to provide supplementation to animals in need of restoring physiological self-generation of water soluble vitamins, demonstrating a need in the art for improved compositions and methods of use.

Accordingly, it is an objective of the claimed invention to formulate improved supplementation compositions to restore an animal's self-generation of water soluble vitamins in order to improve the overall growth, health, immune system and feeding efficiency of ruminant wildlife.

### BRIEF SUMMARY OF THE INVENTION

The present invention describes compositions and methods of using compositions for wildlife nutritional supplementation. The invention describes ruminant feed supplementation compositions capable of enhancing an animal's microbial synthesis of water soluble vitamins to enhance and/or restore an animal's nutritional consumption and overall health.

According to an embodiment of the invention, a method for regenerating a ruminant's synthesis of water soluble vitamins includes detecting a ruminant having decreased self-synthesis of at least one water soluble vitamin, administering a wildlife nutritional supplementation composition and restoring the ruminant's microbial synthesis of said water soluble vitamin(s).

According to a further embodiment of the invention, a method for enhancing the growth and health of wildlife through nutritional supplementation includes detecting a population of deer exhibiting a decrease in microbial synthesis of at least one water soluble vitamin, administering a wildlife nutritional supplementation composition, wherein the composition comprises a source of macrominerals, a source of microminerals, a source of fat soluble vitamins and at least one water soluble vitamin, wherein said deer are provided at least 90% of its daily nutritional requirements through the consumption of said composition, and restoring said deer's microbial synthesis of said water soluble vitamins.

According to a still further embodiment of the invention, a wildlife nutritional supplementation composition includes from about 5-15% calcium, from about 1-4% phosphorus, less than 5% potassium, from about 1-5% sulfur, from about 10-40% sodium, less than 5% magnesium, from about 800-1,600 PPM iron, from about 10-100 PPM copper, from about 60-150 PPM manganese, from about 20-100 PPM zinc, from about 1-50 PPM cobalt, from about 1-50 PPM selenium, from about 1-50 PPM iodine, from about 10,000-100,000 IU/LB Vitamin A, from about 10,000-100,000 IU/LB Vitamin D, from about 20-10,000 IU/LB Vitamin E, from about 40-180 MG/LB biotin, from about 40-100 MG/LB ascorbic acid, from about 20-300 MG/LB folic acid, from about 2-200 MG/LB Vitamin B3, from about 20-500 MG/LB additional water soluble B series vitamin, wherein said water soluble B series vitamin supplementation restores and/or enhances said wildlife's self-synthesis of B vitamins necessary for enhancing growth and health of said wildlife.

### DETAILED DESCRIPTION OF THE PREFERRED EMBODIMENT

Ruminants such as cervid (deer) have structurally different digestive systems which are designed for and enable the microbial synthesis of certain vitamins and amino acids. As a result, the microbial-synthesis of vitamins, namely water soluble vitamins, contributes much more to the total vitamins absorbed in these animals from consumption of food or feed products. Ruminants are designed to generate, via microbial synthesis, all the vitamins it needs, except vitamins A, D, and E (fat soluble vitamins). The fat soluble vitamins are obtained from dietary sources, such as hay or green pasture and other supplementation compositions and exposure to sunlight (for the production of Vitamin D). The bacterial synthesize by intestinal/rumen microbes has long been thought to supply a ruminant animal nearly all of its water soluble vitamins necessary for maintained health and nutrition. See 2007 National Research Counsel of the National Academies, Animal Nutrient Series “The Nutritional Requirements of Small Ruminants,” which is herein incorporated by reference in its entirety. However, according to the unexpected discovery of the present invention, many ruminants have significantly decreased and/or eliminated microbial synthesis of these crucial water soluble vitamins.

The present invention overcomes a significant and unrecognized deficiency in the art. Due to myriad of factors, ruminants in both the wild and penned experience insufficient, or still worse, no microbial synthesis of these crucial vitamins. According to an embodiment of the present invention, ruminant having decreased microbial-synthesis of at least one water soluble vitamin are detected to determine the need for administration of a wildlife nutritional supplementation composition. Beneficially, the wildlife nutritional supplementation composition and methods of use according to the invention, regenerate a ruminant's consumption efficiency and promote and/or restore the animal's ability for microbial synthesis. As a result, the present invention overcomes the problems of unavailable or insufficient feed and food sources, changes in agricultural processes impacting the nutritional content of crops for ruminants, and the levels of microbes in an animal's digestive system.

The embodiments of this invention are not limited to particular compositions and methods for using and administering the compositions as they can vary as are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural references unless the content clearly indicates otherwise. It should also be noted that the term “or” is generally employed in its sense including “and/or” unless the content clearly dictates otherwise. Further, all units, prefixes, and symbols may be denoted in their SI accepted form. Numeric ranges recited within the specification are inclusive of the numbers defining the range and include each integer within the defined range.

Methods of Use and Administration

According to an embodiment of the invention, the wildlife nutritional supplementation composition is used for regenerating a ruminant's synthesis of water soluble vitamins as a result of a nutritional deficiency, stress or other cause in reduction of microbial rumen populations. Preferably, a method for regenerating a ruminant's synthesis of water soluble vitamins according to the invention may comprise, consist of or consist essentially of detecting a ruminant experiencing a decreased microbial-synthesis of at least one water soluble vitamin and administering a wildlife nutritional supplementation composition to restore said ruminant's microbial-synthesis of said water soluble vitamins. According to preferred embodiments of the invention, the ruminant is a deer. Optionally, the methods of the invention also include the measurement of microbes in a ruminant's rumen to determine whether said ruminant can synthesis (and/or the efficiency or extent of synthesis) of water soluble vitamins.

According to an embodiment of the invention the nutritional supplementation composition is administered to the ruminants year-round, during all seasons for a complete and consistent nutritional program. According to the invention, the year-round supplementation promotes greater antler development, enhanced immune system, greater weight gain, enables early consumption by fawns, increases the lactation of birthing does which further supports multiple births and helps to improve the overall genetics of a herd.

According to a further embodiment of the invention, the methods provide a wildlife nutritional supplementation composition providing at least 75% of a ruminant's daily nutritional requirements. The wildlife nutritional supplementation composition preferably comprises at least one water soluble vitamin, which is selected from the group consisting of vitamins C, vitamin B-1 (thiamin), vitamin B-2 (riboflavin), vitamin B-3 (niacin), vitamin B-5 (pantothenic acid), vitamin B-6 (pyridoxine hydrochloride or pyridoxal), vitamin B-7 (biotin or vitamin H), vitamin B-9 (folic acid), vitamin B-12 (cobalamin) and combinations of the same. The wildlife nutritional supplementation composition may also optionally include additional beneficial vitamins and minerals selected from the group consisting of manganese, copper, zinc, iodine, selenium, cobalt, iron, vitamins A, vitamin D, vitamin E, vitamin K, complex vitamins and combinations of the same along with macrominerals selected from the group consisting of calcium, phosphorus, potassium, sulfur, sodium, chloride, magnesium and combinations of the same.

According to a further embodiment of the invention, a method for enhancing the growth and health of wildlife through nutritional supplementation is disclosed. The method may comprise, consist of or consist essentially of detecting a population of deer exhibiting a decrease in microbial synthesis of at least one water soluble vitamin, administering a wildlife nutritional supplementation composition and restoring the deer's microbial synthesis of said water soluble vitamins. The method may further comprise the measuring of microbe population in said deer's rumen that is responsible for synthesis of said water soluble vitamins. One skilled in the art is able to determine a variety of methods for such measuring step, which is not intended to limit the scope of the claimed invention. For example, without intending to place any limitations, the measurement step may include the measuring of both an initial and one or more subsequent measurements of a population for purposes of detecting a trend in microbe population. Moreover, the measurement step may include assaying a limited number of representative animals of a population as opposed to an entire population. Still further, the measurement step may be understood to correlate to the microbe population within an entire herd or population of animals as the animals normally obtain food sources at similar rates and amounts. One of skill in the art will appreciate these expected variations and options present for the measurement step according to the invention.

The method may further comprise restoring the deer's microbial synthesis as determined or measured by either direct and/or indirect markers. For example, an increase in deer's consumption efficiency and/or decrease in deer's feeding requirements from said wildlife nutritional supplementation composition are measurements of restoring the deer's microbial synthesis of said water soluble vitamins according to the invention. Alternatively, a second (or more) measurement of the microbe population within the animal(s) may also be obtained to more definitively define the improvement or restoration of the microbial synthesis within the animal according to the methods of the invention.

According to a preferred embodiment of the invention, the wildlife nutritional supplementation composition comprises a source of macrominerals, a source of microminerals, a source of fat soluble vitamins and at least one water soluble vitamin, wherein said deer are provided at least about 75% of its daily nutritional requirements, preferably at least about 90% of its daily nutritional requirements through the consumption of the wildlife nutritional supplementation composition. More preferably, the wildlife nutritional supplementation composition provides approximately 100% of the deer's daily nutritional requirements. In a still further aspect, the animals may be provided more than 100% of its daily nutritional requirement.

According to an aspect of the invention, the water soluble vitamins are selected from the group consisting of vitamins C, vitamin B-1 (thiamin), vitamin B-2 (riboflavin), vitamin B-3 (niacin), vitamin B-5 (pantothenic acid), vitamin B-6 (pyridoxine hydrochloride or pyridoxal), vitamin B-7 (biotin or vitamin H), vitamin B-9 (folic acid), vitamin B-12 (cobalamin) and combinations of the same. The source of fat soluble vitamins and microminerals in the wildlife nutritional supplementation composition are selected from the group consisting of manganese, copper, zinc, iodine, selenium, cobalt, iron, vitamins A, vitamin D, vitamin E, vitamin K, complex vitamins and combinations of the same. Still further, the source of macrominerals in the wildlife nutritional supplementation composition are selected from the group consisting of calcium, phosphorus, potassium, sulfur, sodium, chloride, magnesium and combinations of the same.

According to the methods of the invention, an animal is administered and/or consumes independently approximately one half ounce to five ounces of said wildlife nutritional supplementation composition on a daily basis. As one skilled in the art will appreciate, the daily consumption needs of an animal may vary according to the species of animal and the conditions the animal is under—including seasonal changes, stress (such as tick infestations, birthing and/or lactation) and the like.

Methods of Making Wildlife Nutritional Supplementation Compositions

According to an embodiment of the invention, the wildlife nutritional supplementation composition results in a synergistic interaction between the formulated vitamins and minerals, causing an interaction that multiplies each ingredient's overall value and contribution to the total formula. Although not intended to be limited to a particular theory of the invention, the methods of making the nutritional supplementation composition, including separating the vitamin and mineral components prior to blending, afford these synergistic reactions.

According to a further embodiment of the invention, the methods of making the wildlife nutritional supplementation composition maintain the quality of the mineral and vitamin supplemental program. The use of all natural food grade components for the vitamins and minerals provides numerous benefits, including for example, promotes consumption by the animals as a result of smaller particulate size providing increased palatability, ingestion and absorption of nutrients. In addition, the use of food grade materials, rather than feed grade ingredients, prevents the deterioration of the components, namely vitamins and minerals. For example, use of feed grade Vitamins A and E results in almost complete deterioration of the vitamins before a product reaches a commercial shelf for sale. According to the present invention, the use of food grade ingredients for the nutritional supplementation composition provides minerals and vitamins with increased shelf life, in particular shelf life and stability are significantly increased when the wildlife nutritional supplementation composition is made according to the methods described herein. The methods of making provide further beneficial results in increased stability in nature (in addition to increased shelf-life). According to the invention, administration of the wildlife nutritional supplementation composition to animals for feed in the wild or penned farms, does not result in immediate deterioration as a result of exposure to weather and the elements. For example, rain, sleet or snow do not denature or deteriorate the compositions of the minerals or vitamins provided in the nutritional supplement composition according to the invention. As a result, year-round administration of the compositions is available.

It is understood that wildlife suffering from mineral, trace mineral and vitamin deficiencies benefit from ongoing, consistent intake of supplements, rather than sporadic consumption. However, often supplements are not stable for the ongoing consumption of the animals, requiring the compositions to be frequently distributed to provide adequate supply. This is the result of a composition becoming ineffective upon prolonged exposure to various environmental conditions and based on the shelf-life of the product. This may further be the result of a composition's inferior formulation exposing the supplementation elements to oxidative or other degradative effects, resulting in products with a shortened shelf-life and inability to provide the desired therapeutic benefits. The methods and compositions according to the present invention overcome this shortfall of the prior art, providing wildlife supplements that are intended for ongoing, consistent intake by animals. Further, the compositions are made according to methods providing enhanced stability to ensure that elements formulated in a supplementation composition are effective in achieving its intended nutritive effects. The compositions and methods of making the compositions according to the invention overcome a significant deficit found in the prior art at the time the present invention was made, demonstrating a solution to a market demand that was not obvious to those of ordinary skill in the pertinent art.

According to an embodiment of the invention, the wildlife nutritional supplement compositions are made according to novel formulation methods. Although not intended to be limited according to a single description of the formulation methods and/or theories for its unexpected formulation benefits, the formulation methods are blended in a manner providing synergistic reactions between and among the compositions' ingredients. The synergistic reactions, including the improved effects of the vitamin ingredients, provide an enhanced wildlife nutritional supplement. The beneficial results include wildlife supplementation compositions providing enhanced nutritive benefits, such as immune system improvements. These results may be demonstrated, for example, through improved repellency of ticks, mosquitoes, gnats, flies and other insects, indicative of improved effectiveness of an administered nutritional supplementation according to the invention.

According to one embodiment, the composition is made by blending the following ingredients: macrominerals, trace minerals and vitamins. According to embodiments of the invention, macrominerals may include for example calcium, phosphorus, potassium, sulfur, sodium, chloride, magnesium and combinations of the same. Macrominerals may be provided in a variety of commercially available and known formulations, including in salt and other forms, which are known to a person having ordinary skill in the art to which the invention pertains. According to an embodiment of the invention, trace minerals may include manganese, copper, zinc, iodine, selenium, cobalt, iron and combinations of the same. According to the various embodiments of the invention, trade minerals may be provided in various forms as may be commercially available and known to a person having ordinary skill in the art to which the invention pertains, including for example salt and other forms.

According to a further embodiment of the invention, vitamins may include Vitamins A, D, E, B1, B2, B3, B5, B6, B7, B9, B12, C, K, complex vitamins and other essential and non-essential vitamins and combinations of the same in various known forms. According to the invention, dietary nutrients including macrominerals, trace minerals and vitamins may further include fatty acids, amino acids, dietary minerals and other elements as may be ascertained by a skilled artisan as providing benefit for wildlife supplementation and therefore may be utilized according to the methods and compositions of the present invention. According to a further embodiment of the invention, the various dietary nutrients may include all organic formulations.

According to one embodiment, the methods of making a wildlife nutritional supplement include the addition of one or more additive mixtures to a macromineral formulation. According to a preferred embodiment, a two-part additive mixture is added to a macromineral formulation. According to embodiments of the invention, the two-part additive mixture may comprise a first additive mixture providing desired amounts of calcium, copper, iron, magnesium, zinc and dextrose. The two-part additive mixture may further comprise a second additive mixture providing desired amounts of vitamins, iodine and dextrose. Alternatively, according to the invention, the two-part additive mixture may comprise a first additive mixture providing desired amounts of vitamins, iodine and dextrose, with a second additive mixture providing desired amounts of calcium, copper, iron, magnesium, zinc and dextrose.

According to a preferred embodiment the additive mixtures are continuously blended with the macromineral source. According to a still further preferred embodiment the additive mixtures are continuously blended with the macromineral source for at least about 5 minutes, preferably at least about 10 minutes, and more preferably for at least about 15 minutes. One skilled in the art to which the invention pertains will ascertain means by which the blending described herein may occur. For example, a paddle-blender may be used according to the invention to first blend the macrominerals.

According to a preferred embodiment, the heaviest component of the supplementation formulation, the macrominerals, are first blended to ensure there is an even dispersement of the macromineral components. Preferably, there is a homogenous mixture of the macromineral components. According to a preferred embodiment, one of the additive mixtures is next added to the blended macrominerals and continuously blended before the addition of the second additive mixture. According to an embodiment the additive mixtures may be either a mineral additive (referred to as either a micromineral or trace mineral) or vitamin additive. Preferably, the macrominerals and two additive mixtures are continuously blended for at least about 15 minutes.

Although not intending to be limited according to a particular theory for the unexpected and synergistic results obtained according to the invention, the results are hypothesized to result from the blending of the supplement's ingredients as described herein. According to the invention, the final blending of a multi-part additive mixture maintains separation of essential elements and vitamins prior to formulation. It is demonstrated that the separation of certain vitamins from elements having a potentially degradative or oxidative effect on the supplementation composition results in unexpectedly enhanced performance of the vitamins, including synergistic results as shown by the efficacy of the animal nutritional supplementation.

According to one embodiment of the invention, the separation of additive mixtures comprising the oxidative elements, such as copper, from vitamins, such as B series vitamins, prevents the onset of decomposition of the desired vitamins within the formulations. According to a theory of the invention, elemental minerals such as copper may catalyze the oxidation of vitamins, such as for example, vitamins B and/or C. Further, the elemental minerals may accelerate the decomposition of the vitamins if maintained in the formulation for extended periods of time. Therefore, according to the invention, to achieve a nutritional supplementation composition having enhanced effects, the addition of the minerals and vitamins are made from separately stored and manufactured additive mixtures.

According to the invention, the blending of at least two additive mixtures with a macromineral formulation promotes the stability and shelf-life of the wildlife supplementation composition. According to a further embodiment of the invention, blending techniques described herein promote the synergistic reaction of “B” series vitamins. Without the potentially harmful effects of any oxidative minerals, the vitamin B elements of a supplementation formulation are able to provide the desired result of enhanced immune system protection, through the repellency of ticks, gnats and other insects. The results observed for protecting wildlife from such insects is synergistic in comparison to a product that does not follow the blending techniques according to the invention.

Accordingly, products administered to free choice fed ruminants and other animals provide sufficient amounts of the nutritive value provided by a composition in order to effectuate the desired nutritional outcomes. This unexpected result of enhancing a product's stability to promote the desired nutritional outcomes in wildlife unexpectedly results from the modification of the formulation methods. It was not obvious to one skilled in the art that the manufacture or purchase of separate additive mixtures for minerals and vitamins would enhance the nutritive value and effects in wildlife, as well as provide a composition having an improved shelf-life due to enhanced product stability. According to an embodiment of the invention, the compositions providing such enhanced nutritive value and effects have a shelf-life of at least two years while maintaining potency and efficacy. These beneficial effects unexpectedly result from the prevention of degradation and oxidation of vitamins and other components found in the supplementation composition according to the invention.

According to an embodiment of the invention, the wildlife nutritional supplement composition provides enhanced formulation due to the methods of making the same. This provides unexpected and superior results over prior art supplementation compositions, which are limited by their formulations which combine all of the minerals and nutrients with vitamins. As a result, such products do not provide compositions having an improved and uniform blend of products, products with sufficient shelf-life, nor products demonstrating a synergistic reaction of vitamins contained in the formulation. As described according to the present invention, the formulation and methods of making the same provide enhanced supplementation compositions having improved immune system benefits and nutritive benefits as a result of the preferred blending methods described herein with regard to the core minerals and vitamins.

Wildlife Nutritional Supplementation Compositions

According to an embodiment of the invention the nutritional supplementation composition provides food grade vitamins and minerals. The beneficial use of food grade quality components and formulas promotes the restoration of microbial populations for microbial synthesis of water soluble vitamins and amino acids, promotes weight gain, improves antler growth, improves health and immune system function along with beneficially controlling tick infestations. Importantly the nutritional supplementation composition provides at least 75%, 80%, 85%, 90%, 95% and most preferably 100% of the total required daily minerals and vitamins for nutritional needs of ruminant animals. According to a further embodiment of the invention, the nutritional supplementation composition provides at least 75%, 80%, 85%, 90%, 95% and most preferably 100% of the total required daily minerals and vitamins for nutritional needs of white tailed deer. In still further aspects of the invention the nutritional supplementation composition provides more than 100% of the total required daily minerals and vitamins for nutritional needs of the particular animal population. Beneficially the nutritional supplementation composition further contains high quality ingredients, including food grade ingredients rather than feed grade ingredients.

According to an embodiment of the invention, the nutritional supplementation composition provides supplementation for minerals and water soluble vitamins particularly for white tailed deer (both penned and wild). The wildlife nutritional supplementation composition preferably contains at least 10 different minerals and 10 different vitamins, more preferably the wildlife nutritional supplementation composition preferably contains at least 14 different minerals and 12 different vitamins. According to an embodiment of the invention, the wildlife nutritional supplementation composition contains vitamins and minerals selected from the group consisting of calcium, phosphorus, salt/chlorine, sodium, magnesium, potassium, sulfur, iron, copper, manganese, zinc, cobalt, selenium, iodine, vitamins A, D, E, B (folic acid), B3 (niaciamide), H (biotin), C, B1, B2, B5, B6 and B12. According to an embodiment of the invention the nutritional supplement composition maintains a healthier immune system for animals.

According to an embodiment of the invention, the wildlife nutritional supplementation composition comprises from about 5-15% calcium, from about 1-4% phosphorus, less than 5% potassium, from about 1-5% sulfur, from about 10-40% sodium, less than 5% magnesium, from about 800-1,600 PPM iron, from about 10-100 PPM copper, from about 60-150 PPM manganese, from about 20-100 PPM zinc, from about 1-50 PPM cobalt, from about 1-50 PPM selenium, from about 1-50 PPM iodine, from about 10,000-100,000 IU/LB Vitamin A, from about 10,000-100,000 IU/LB Vitamin D, from about 20-10,000 IU/LB Vitamin E, from about 40-180 MG/LB biotin, from about 40-100 MG/LB ascorbic acid, from about 20-300 MG/LB folic acid, from about 2-200 MG/LB Vitamin B3, from about 20-500 MG/LB additional water soluble B series vitamin, wherein said water soluble B series vitamin supplementation restores and/or enhances said wildlife's self-synthesis of B vitamins necessary for enhancing growth and health of said wildlife. According to a further embodiment of the invention, the B series vitamins are selected from the group consisting of pantothenic acid, riboflavin, niacin, thiamine, cobalamin, pyridoxine hydrochloride, similar water soluble vitamins and combinations of the same.

According to an embodiment of the invention, the wildlife nutritional supplement composition includes decreased levels of calcium and phosphorus (compared to various commercially-available formulations), increased levels of sodium and salts to create a more palatable product to animals to promote consumption, and “B” and “C” series vitamins to maintain the overall health of ruminant animals. The supplement composition may comprise levels of calcium below 20% and phosphorus below 10%, preferably amounts ranging between 5-19.6% calcium and 4%-9.8% phosphorus. According to a preferred embodiment, the inclusion of “B” series vitamins provides vitamin sources previously believed to be obtainable by wildlife through natural resources. However, such ideal environmental conditions and natural resources are not always present, particularly in arid, dry regions. According to a further embodiment of the invention, the nutritional supplement compositions provides “B” vitamin supplementation necessary to protect wildlife from insect bites, such as ticks, and enhances the animal's immune system strength.

According to a further embodiment of the invention, the compositions include anthelminthic (also referred to as de-worming) agents. According to a preferred embodiment the anthelminthic agent is fenbendazole. The benefits of including anthelminthic agents are effective for treating and prophylactically preventing infectious agents such as the various species of gastrointestinal nematodes known to infect wildlife, such as white-tailed deer and livestock. See e.g., Prestwood, A. K., et al., Gastrointestinal nematodes, p. 318-350 (1981); Davidson, et al., Diseases and parasites of white-tailed deer. Tall Timbers Res. Sta. Misc. Pub. 7; and Schultz, et al., Efficacy of fenbendazole against gastrointestinal nematodes in white-tailed deer. Journal of Range Management. 46:240-244 (1993). These references are incorporated herein by reference in their entirety.

According to a further embodiment of the invention, the compositions include antibiotic agents. Inclusion of an antibiotic may provide similar benefits to the periodic inducement into a human diet, such that the animals are exposed to prophylactic and regulating effects of antibiotics. According to a preferred embodiment the antibiotic agent is oxytetracycline or similar members of the tetracycline class of antibiotics. According to further embodiments of the invention, additional classes of antibiotics may be included in the formulations as may be readily ascertained by one of ordinary skill in the art to which the invention pertains. The inclusion of an antibiotic in the nutritional supplementation composition allows the animal to use what it needs and discard the remaining antibiotic through its natural waste system.

The wildlife nutritional supplement composition according to the invention may be formulated into a granular or block form. According to an alternative embodiment the wildlife nutritional supplement composition according to the invention may be formulated into a liquid medium.

According to an embodiment of the invention, the nutritive supplementation composition may comprise from about 5-15% calcium, from about 1-10% phosphorus, less than 5% potassium, from about 1-5% sulfur, from about 10-40% sodium and less than 5% magnesium. According to a further embodiment, the composition may further comprise from about 800-1,600 PPM iron, from about 10-100 PPM copper, from about 60-150 PPM manganese, from about 20-100 PPM zinc, from about 1-50 PPM cobalt, from about 1-50 PPM selenium, from about 1-50 PPM iodine, from about 10,000-100,000 IU/LB Vitamin A, from about 10,000-100,000 IU/LB Vitamin D, from about 20-10,000 IU/LS Vitamin E, from about 40-180 MG/LB biotin and from about 40-100 MG/LB ascorbic acid.

According to an alternative embodiment of the invention, the nutritive supplementation formulation may comprise from about 7.5-8.5% calcium, about 3.5% phosphorus, about 32-37% salt, at least one “B” series vitamin is selected from a group consisting of pantothenic acid, folic acid, riboflavin, niacin, thiamine, cobalamin, and pyridoxine hydrochloride, about 16-19% sodium, about 0.15% magnesium, about 0.15% potassium, about 2.5% sulfur, about 1,200 PPM iron, about 20 PPM copper, about 105 PPM manganese, about 45 PPM zinc, about 5 PPM cobalt, about 1 PPM selenium, about 1 PPM iodine, about 50,000 IU/LB Vitamin A, about 20,000 IU/LB Vitamin D, about 50 IU/LB Vitamin E, about 134 MG/LB biotin, about 60 MG/LB ascorbic acid, oxytetracycline and fenbendazole.

Alternate embodiments may comprise from about 17-18% calcium, about 9% phosphorus, about 11-12% salt, at least one “B” series vitamin selected from a group consisting of pantothenic acid, folic acid, riboflavin, niacin, thiamine, cobalamin, and pyridoxine hydrochloride, about 10-11% sodium, about 3.7% magnesium, about 3.7% potassium, about 0.0037% sulfur, about 1,666 PPM iron, about 185 PPM copper, about 60 PPM manganese, about 1,388 PPM zinc, about 2 PPM cobalt, about 0.57 PPM selenium, about 13 PPM iodine, about 50,000 IU/LB Vitamin A, about 20,000 IU/LB Vitamin D, about 50 IU/LB Vitamin E, about 134 MG/LB biotin, about 60 MG/LB ascorbic acid, oxytetracycline and fenbendazole.

According to an embodiment, the formulation methods and compositions according to the invention provide an enhanced wildlife nutritional supplement that retains stability to provide the complete intended supplementation to such animal. Further, according to an embodiment, the formulation methods and compositions according to the invention described herein provide enhanced immune function of wildlife animals consuming the supplementation compositions. According to one embodiment, the supplementation compositions provide treatment and/or prophylaxis from various infectious agents, including for example species of gastrointestinal nematodes.

Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology is used in accordance with the definitions set out below.

The term “about,” as used herein, refers to variation in the numerical quantity that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like. The term “about” also encompasses amounts that differ due to different equilibrium conditions for a composition resulting from a particular initial mixture. Whether or not modified by the term “about”, the claims include equivalents to the quantities referred and variations in the numerical quantities that can occur, for example, through typical measuring and liquid handling procedures used for making concentrates or use solutions in the real world; through inadvertent error in these procedures; through differences in the manufacture, source, or purity of the ingredients used to make the compositions or carry out the methods; and the like.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

All publications and patent applications in this specification are indicative of the level of ordinary skill in the art to which this invention pertains. All publications and patent applications are herein incorporated by reference to the same extent as if each individual publication or patent application was specifically and individually indicated by reference.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments of the invention, in addition to those shown and described herein, will be apparent to those skilled in the art from the foregoing description. Such modifications are also intended to fall within the scope of the appended claims.

### Example 1

Studies analyzing the effectiveness of these approaches reveal substantial and unexpected advantages as described herein for example. It is known that calcium and phosphorus are essential components in wildlife nutritional supplements, often present in amounts ranging between 5-19.6% calcium and a minimum of 4% phosphorus in order to provide the nutritionally beneficial effects. References have shown that decreasing the concentration of these minerals promotes the intake or consumption of a supplementation product. For example, a formulation with calcium and phosphorus concentrations below 5-19.6% and 4%, respectively, results in increased total consumption by wildlife due to palatability and other desirable attributes through the addition of sodium and salt to act as an attractant for wildlife. This results in increased regular consumption and year-round usage by wildlife providing an overall greater intake of calcium and phosphorus than was previously achieved by products comprising greater concentrations of the same minerals. The reduction of calcium and phosphate further enables the addition of “B” series vitamins to the formulation.

However, the approach to formulating supplementation products with decreased levels of calcium and phosphorus, in combination with enhanced use of sodium and salts, will only result in improved animal health to the extent the product's formulation is stable and compatible with its formulation components. As described according to the methods of making the claimed nutritional supplementation composition, formulations separating the micromineral and vitamin additives result in a product having increased shelf-life. Accordingly, wildlife are provided a product with desirable nutritive content that remains stable to provide the intended benefits.

### Example 2

A vitamin premix, containing iodine, for the nutritional supplement according to the invention is provided (on a per gram basis):

| | | |
|---|---|---|
| Palmitate | 50,000 | IU |
| Cholecalciferol | 20,000 | IU |
| dl-alpha-tocopheryl acetate | 50 | IU |
| Biotin | 0.3 | mg |
| Folic Acid | 0.4 | mg |
| Niacin | 20 | mg |
| Calcium d-Pantothenate | 10 | mg |
| Thiamin mononitrate | 1.5 | mg |
| Cyanocobalamin | 8 | mcg |
| Riboflavin | 1.7 | mg |
| Pyridoxine hydrochloride | 2 | mg |
| Ascorbic acid | 60 | mg |
| Potassium iodide | 0.15 | mg |

| | |
|---|---|
| Dextrose | Q.S. gram |

The vitamin premix formulation provides a 24 month shelf-life for the nutrients in the premix formulation.

### Example 3

A mineral premix, containing the copper content for the nutritional supplement according to the invention is provided (on a per gram basis):

| | |
|---|---|
| Calcium carbonate | 0.45 mg |
| Copper sulfate | 0.03 mg |
| Iron | 0.003 mg |
| Magnesium oxide | 0.07 mg |
| Zinc oxide | 0.003 mg |
| Dextrose | Q.S. gram |

The mineral premix formulation provides a 24 month shelf-life for the nutrients in the premix formulation.

### Example 4

The wildlife nutritional supplementation composition according to the invention provides measurable benefits over control (no supplementation or feed, such as corn). Penned white tailed deer herds were fed the wildlife nutritional supplementation composition according to the invention. The wildlife nutritional supplementation compositions provided the animals 100% of total required daily minerals and vitamins for nutritional needs of white tailed deer. Beneficially the nutritional supplement composition further contains high quality ingredients, including food grade ingredients rather than feed grade ingredients. The control group was fed a standard diet, such as corn.

The table below demonstrates the control arm: corn nutrients compared to the nutritional needs of deer.

| | Corn | Nutritional Needs |
|---|---|---|
| % Protein | 9 | 14+ |
| % Calcium | 0.03 | 0.6+ |
| % Phosphorous | 0.3 | 0.3+ |
| Copper, PPM | 4 | 20+ |
| Zinc, PPM | 19 | 60+ |
| Selenium, PPM | 0.13 | 0.3+ |
| Digestible Energy | 80% | 55%+ |

The wildlife nutritional supplementation composition was provided in the form of loose granules in order to provide the macro and micronutrients, including minerals and vitamins. The loose granular formulation was preferred over block compositions. The loose granular mineral and vitamin compositions according to the invention are more readily absorbed and used as it is more natural for wildlife to consume nutrients from the ground. Animals demonstrated a preference for the loose granular formulation. In addition, the loose granular supplementation compositions are preferred over blocks as a result of the longer degradation time required for blocks to break down in nature and provide the nutrients for wildlife. The loose granular compositions provide the wildlife immediate and full advantage of the minerals and vitamins for consumption.

A before and after comparison was made between white tailed deer obtained from the same initial genetic pool, raised at the same farm under Department of Conservation control. Deer were killed after 3.5 years and the animals feed the nutritional supplementation composition according to the invention demonstrated an average increase in weight to the control group of from about 35-50+ pounds for does and from about 75-100+ pounds for bucks. The results (obtained in the form of photographs and physical measurements) demonstrate that the nutritional supplement composition provided benefits including enhanced weight gain, greater antler development, better overall health and immune response.

### Example 5

Feeding frequency of the wildlife nutritional supplementation composition according to the invention was analyzed. As one skilled in the art appreciates, stress is the single largest factor on wildlife growth and premature death. White tailed deer go through several stress cycles each year (i.e. change in seasons, fertility, tick infestations, etc.) and therefore the benefits of the wildlife nutritional supplementation composition according to the invention were speculated to be most efficacious when administered year round.

White tailed deer in the pen and wild were provided maintained and constant access to the wildlife nutritional supplementation composition and consumption was monitored. Animals demonstrated an increase in consumption (as measured by visits and feeding from feed locations) during increased stress conditions. It was observed that animals fed until dietary consumption needs were met. Pen fed deer were observed to consume daily supplementation, whereas wild deer fed more in non-crop growing times of year (in effort to supplement in more seasonal patterns). The results of the observed studies showed that less than about 5 ounces, preferably from about 1-2 ounces of the wildlife nutritional supplementation composition per day were required. As a result, the present invention demonstrated that deer met its nutritional needs and achieved the most efficient consumption after feeding on approximately 1-2 ounces of the wildlife nutritional supplementation composition per day, year round.

In addition, the results of the feeding frequency demonstrate that year-round supplementation promotes greatest increase in antler development, enhanced immune system, weight gain and improving the overall genetics of a herd. Benefits were achieved from early, consistent consumption by fawns as well as greater lactation by birthing does in order to support multiple births as a result of being fed the nutritional supplement composition according to the invention.

1. A method for making a nutritional supplement product comprising:
blending a macromineral composition with a first additive composition to form a first nutritional supplement composition;
forming a second additive composition;
blending the first nutritional supplement composition and the second additive composition to form the nutritional supplement product;
wherein the nutritional supplement product comprises from about 5-19.6% calcium, from about 1-10% phosphorus, less than 5% potassium, from about 1-5% sulfur, from about 10-40% sodium and less than 5% magnesium.

2. The method of claim 1 wherein the nutritional supplement product further comprises from about 800-1,600 PPM iron, from about 10-100 PPM copper, from about 60-150 PPM manganese, from about 20-100 PPM zinc, from about 1-50 PPM cobalt, from about 1-50 PPM selenium, from about 1-50 PPM iodine, from about 10,000-100,000 IU/LB Vitamin A, from about 10,000-100,000 IU/LB Vitamin D, from about 20-10,000 IU/LB Vitamin E, from about 40-180 MG/LB biotin, from about 40-100 MG/LB ascorbic acid and at least one B series vitamin.

3. The method of claim 2 wherein said B series vitamin is selected from the group consisting of pantothenic acid, folic acid, riboflavin, niacin, thiamine, cobalamin, pyridoxine hydrochloride and combinations of the same.

4. The method of claim 2 wherein the B series vitamin includes from about 20-300 MG/LB folic acid, from about 2-200 MG/LB Vitamin B3, from about 20-500 MG/LB additional water soluble B series vitamin.

5. The method of claim 1 wherein said first additive composition is a trace mineral composition comprising minerals selected from the group consisting of manganese, copper, zinc, iodine, selenium, cobalt, iron and combinations of the same and said second additive composition is a vitamin composition comprising vitamins selected from the group consisting of Vitamins A, D, E, B1, B2, B3, B5, B6, B7, B9, B12, C, K, complex vitamins and combinations of the same.

6. The method of claim 1 wherein said macromineral composition comprises macrominerals selected from the group consisting of calcium, phosphorus, potassium, sulfur, sodium, chloride, magnesium and combinations of the same.

7. The method of claim 1 wherein the nutritional supplement product further comprising an antibiotic.

8. The method of claim 1 wherein the nutritional supplement product further comprising an anthelminthic component.

9. The method of claim 1 wherein the nutritional supplement product comprises from about 5-15% calcium.

10. A method for making a nutritional supplement product comprising:
blending a macromineral composition with a first additive composition to form a first nutritional supplement composition;
forming a second additive composition;
blending the first nutritional supplement composition and the second additive composition to form the nutritional supplement product;
wherein the nutritional supplement product comprises from about 5-19.6% calcium, from about 1-10% phosphorus, less than 5% potassium, from about 1-5% sulfur, from about 10-40% sodium and less than 5% magnesium, about 800-1,600 PPM iron, from about 10-100 PPM copper, from about 60-150 PPM manganese, from about 20-100 PPM zinc, from about 1-50 PPM cobalt, from about 1-50 PPM selenium, from about 1-50 PPM iodine, from about 10,000-100,000 IU/LB Vitamin A, from about 10,000-100,000 IU/LB Vitamin D, from about 20-10,000 IU/LB Vitamin E, from about 40-180 MG/LB biotin, from about 40-100 MG/LB ascorbic acid and at least one B series vitamin.

11. The method of claim 10 wherein said B series vitamin is selected from the group consisting of pantothenic acid, folic acid, riboflavin, niacin, thiamine, cobalamin, pyridoxine hydrochloride and combinations of the same.

12. The method of claim 10 wherein the B series vitamin includes from about 20-300 MG/LB folic acid, from about 2-200 MG/LB Vitamin B3, from about 20-500 MG/LB additional water soluble B series vitamin.

13. The method of claim 10 wherein said first additive composition is a trace mineral composition comprising minerals selected from the group consisting of manganese, copper, zinc, iodine, selenium, cobalt, iron and combinations of the same and said second additive composition is a vitamin composition comprising vitamins selected from the group consisting of Vitamins A, D, E, B1, B2, B3, B5, B6, B7, B9, B12, C, K, complex vitamins and combinations of the same.

14. The method of claim 10 wherein said macromineral composition comprises macrominerals selected from the group consisting of calcium, phosphorus, potassium, sulfur, sodium, chloride, magnesium and combinations of the same.

15. The method of claim 10 wherein the nutritional supplement product further comprising an antibiotic.

16. The method of claim 10 wherein the nutritional supplement product further comprising an anthelminthic component.

17. The method of claim 10 wherein the nutritional supplement product comprises from about 5-15% calcium.

18. A method for making a homogeneous granular nutritional supplement product comprising: forming a macromineral composition by mixing a plurality of macromineral components;
blending the macromineral composition with a trace mineral composition to form a first nutritional supplement composition;
forming a vitamin composition by mixing a plurality of vitamins;
blending the first nutritional supplement composition and the vitamin composition to form the homogeneous granular nutritional supplement product;
wherein the homogeneous granular nutritional supplement product comprises from about 5-19.6% calcium, from about 1-10% phosphorus, less than 5% potassium, from about 1-5% sulfur, from about 10-40% sodium and less than 5% magnesium.

19. The method of claim 18 wherein the homogeneous granular nutritional supplement product further comprises from about 800-1,600 PPM iron, from about 10-100 PPM copper, from about 60-150 PPM manganese, from about 20-100 PPM zinc, from about 1-50 PPM cobalt, from about 1-50 PPM selenium, from about 1-50 PPM iodine, from about 10,000-100,000 IU/LB Vitamin A, from about 10,000-100,000 IU/LB Vitamin D, from about 20-10,000 IU/LB Vitamin E, from about 40-180 MG/LB biotin, from about 40-100 MG/LB ascorbic acid and at least one B series vitamin.

20. The method of claim 18 wherein the trace mineral composition comprises minerals selected from the group consisting of manganese, copper, zinc, iodine, selenium, cobalt, iron and combinations of the same and said vitamin composition comprises vitamins selected from the group consisting of Vitamins A, D, E, B1, B2, B3, B5, B6, B7, B9, B12, C, K, complex vitamins and combinations of the same.

21. The method of claim 18 wherein said macromineral composition comprises macrominerals selected from the group consisting of calcium, phosphorus, potassium, sulfur, sodium, chloride, magnesium and combinations of the same.

22. The method of claim 18 wherein the nutritional supplement product comprises from about 5-15% calcium.
